(19) United States
(12) Patent Application Publication
(10) Pub. No.: US 2016/0033487 A1
(43) Pub. Date: Feb. 4, 2016

(54) DISPENSING DEVICE, IMMUNOASSAY ANALYZER AND METHOD THEREOF

(71) Applicant: SHENZHEN MINDRAY BIO-MEDICAL ELECTRONICS CO., LTD., Shenzhen (CN)

(72) Inventors: Zhen Zhang, Shenzhen (CN); Peng Zhou, Shenzhen (CN); Jun Wang, Shenzhen (CN); Chuanfen Xie, Shenzhen (CN)

(73) Assignee: SHENZHEN MINDRAY BIO-MEDICAL ELECTRONICS CO., LTD.

(21) Appl. No.: 14/885,901

(22) Filed: Oct. 16, 2015

Related U.S. Application Data
(63) Continuation of application No. PCT/CN2013/083961, filed on Sep. 23, 2013.

(30) Foreign Application Priority Data
Apr. 16, 2013 (CN) 201310132100.5

(51) Int. Cl. G01N 33/53; G01N 33/536; G01N 35/10; G01N 35/00; G01N 35/02

(52) U.S. Cl. CPC G01N 33/5304; G01N 35/00871; G01N 35/025; G01N 35/1065; G01N 35/1002; G01N 33/536; G01N 2035/0444

(57) ABSTRACT

Dispensing devices and methods are provided. The dispensing devices can include a first dispensing station arranged on a periphery of a holding unit for dispensing a first reaction component, and a second dispensing station arranged on the holding unit for dispensing a second reaction component. The first reaction component and the second reaction component are dispensed. Operations on the holding unit are not affected by dispensing of the first reaction component, improving test efficiency. Because reaction containers do not need to be transferred out of the holding unit to dispense the second reaction component, test processes are simplified, In addition, the dispensing unit is not restricted to being arranged around the holding unit, and the first dispensing unit and second dispensing unit are arranged separately, so restriction of and interference with the dispensing units are avoided.

### TECHNICAL FIELD

The present disclosure relates to medical instrument technologies, and particularly to a dispensing device, immunoassay analyzer and method thereof.

### SUMMARY

Disclosed herein are dispensing devices, immunoassay analyzers and methods thereof.

A first aspect of the present disclosure is a dispensing device. The dispensing device includes: a holding unit that holds and transfers one or more reaction containers along a transfer path of the holding unit; a first dispensing station that is arranged on a periphery of the holding unit; a second dispensing station that is arranged on the holding unit; a transfer unit that transfers the one or more reaction containers between the first dispensing station and the holding unit on which the second dispensing station is arranged; a dispensing unit that dispenses a first reaction component to the reaction containers located at the first dispensing station at a dispensing position of the first dispensing station, and dispenses a second reaction component to the reaction containers located at the second dispensing station; and a control unit coupled to the transfer unit, the holding unit and the dispensing unit. The control unit controls the transfer unit to transfer the one or more reaction containers between the first dispensing station and the holding unit on which the second dispensing station is arranged, and controls the dispensing unit to dispense the first reaction component to the reaction containers located at the first dispensing station and dispense the second reaction component to the reaction containers located at the second dispensing station.

A second aspect of the present disclosure is a dispensing method. The dispensing method includes: transferring by a transfer unit one or more reaction containers to a dispensing position of a first dispensing station which is arranged on a periphery of a holding unit; dispensing by a dispensing unit a first reaction component to the one or more reaction containers located at the first dispensing station; transferring by the transfer unit the one or more reaction containers from the first dispensing station to a second dispensing station which is arranged on the holding unit; and dispensing by the dispensing unit a second reaction component to the reaction containers located at the second dispensing station.

A third aspect of the present disclosure is an immunoassay analyzer. The immunoassay analyzer includes: a dispensing device of the first aspect of the present disclosure; a wash unit arranged on the periphery of the holding unit, wherein the wash unit removes an unbound component from the one or more reaction containers and adds a signal reagent which can react with reaction complexes formed by the first reaction component and the second reaction component in the reaction containers for generating an optical signal; and an optical detection unit, which is arranged on the periphery of the holding unit and detects the optical signal from the reaction containers located at the holding unit.

A fourth aspect of the present disclosure is an immunoassay method. The immunoassay method includes: transferring by a transfer unit a reaction container to a first dispensing station which is arranged on a periphery of a holding unit; dispensing by a dispensing unit a first reaction component to the reaction container located at the first dispensing station; transferring by the transfer unit the reaction container from the first dispensing station to a second dispensing station arranged on the holding unit; dispensing by the dispensing unit a second reaction component to the reaction container located at the second dispensing station; transferring by the holding unit the reaction container which is dispensed with the first reaction component and the second reaction component; transferring by the holding unit the reaction container that holds the first reaction component and the second reaction component for an incubation time to a transfer station of the holding unit; transferring by the transfer unit the reaction container from the transfer station to a wash unit for removing an unbound component from the reaction container; adding a signal reagent that reacts with reaction complexes formed by the first reaction component and the second reaction component within the reaction container to generate an optical signal; and transferring by the holding unit the reaction container added with the signal reagent to a detection station of the holding unit, wherein the optical signal from the reaction container is detected by an optical detection unit arranged on the periphery of the holding unit.

### BRIEF DESCRIPTION OF THE DRAWINGS

FIG. 1 is a schematic diagram illustrating principles of detecting a sample by an immunoassay analyzer;

FIG. 2 is a schematic diagram illustrating a first test process of an immunoassay analyzer using a one-step protocol;

FIG. 3 is a schematic diagram illustrating a second test process of an immunoassay analyzer using a one-step protocol;

FIG. 4 is a schematic diagram illustrating a test process of an immunoassay analyzer using a two-step, one-bound-free (B/F) protocol;

FIG. 5 is a schematic diagram illustrating a test process of an immunoassay analyzer using a two-step, two-B/F protocol;

FIG. 6 is a schematic diagram of a dispensing device according to a first embodiment of this disclosure;

FIG. 7 is a schematic diagram of a dispensing device according to a second embodiment of this disclosure;

FIG. 8 is a schematic diagram of a dispensing device according to a third embodiment of this disclosure;

FIG. 9 is a schematic diagram of a dispensing device according to a fourth embodiment of this disclosure;

FIG. 10 is a flow chart for a dispensing method according to an embodiment of this disclosure;

FIG. 11 is a schematic diagram of an immunoassay analyzer according to an embodiment of this disclosure;

FIG. 12 is a flow chart for an immunoassay method according to a first embodiment of this disclosure;

FIG. 13 is a flow chart for an immunoassay method according to a second embodiment of this disclosure; and

FIG. 14 is a flow chart for an immunoassay method according to a third embodiment of this disclosure.

### DETAILED DESCRIPTION

An immunoassay analyzer is usually used to detect target analyte of blood, urine or other body liquids in clinical laboratories. The immunoassay analyzer supports several kinds of reaction modules, including a competitive module, a sandwich module, an indirect module and a capture module. An immunoassay method mainly includes radioimmunoassay (RIA), enzyme linked immunosorbent assays (ELISA) and luminescence immunoassay (LIA). Because the luminescence immunoassay has advantages of environmental protection, high detection sensitivity and wide test range, it has become a main clinical immunoassay. Luminescence immunoassay is a new labeling immune analysis method which is set up by combining luminescence analysis and immune reaction, and it correspondingly combines high sensitivity of chemiluminescence and high specificity of immune reaction.

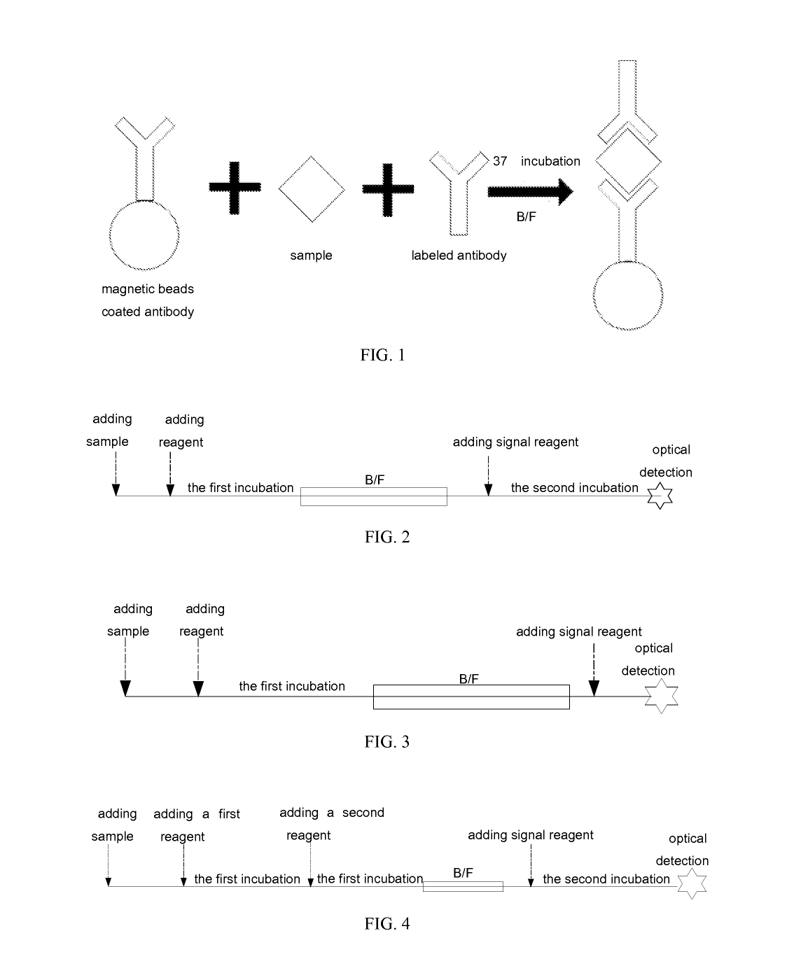

For a one-step sandwich protocol, referring to FIG. 1, when detecting an object substance in a sample, a magnetic bead reagent is formed by coating the magnetic bead with an antibody/antigen, and a labeling reagent is formed by marking an antibody with a given marker. There are usually multiple reagent components for analyzing a certain kind of analytical item, e.g., magnetic bead reagent components, marking reagent components, etc. Different components of the reagent can be held in different containers or in different cells of the same reagent container. During testing, the sample is mixed together with the magnetic bead reagent, the labeling reagent and other reagents to form a reaction solution in a reaction container. In certain conditions, reaction complexes are formed by those sample and reagents through an incubation reaction. Then, unbound components of markers, reagents and the sample are removed by bound-free (B/F) technology. Thereafter, a signal reagent is added for reacting with a label/marker of the reaction complexes to generate light. The signal reagent may include one or more types, e.g., a luminescent substrate solution, a pre-excitation liquid, an excitation liquid, a luminescence enhancement solution, etc. There are many techniques for specific coating and B/F in addition to the magnetic bead method. For example, in another method, the antibody is coated on a wall of the reaction container or plastic beads, etc.

The immunoassay analyzer needs to support several different kinds of test protocols corresponding to different characteristics of the test items.

(1) One-Step Protocol

Referring to FIG. 2, a one-step protocol is the simplest protocol. A reagent (which may include several kinds of compositions) and a sample are added to a reaction container and mixed together so as to form a reaction solution. Thereafter, a B/F process is performed after the reaction solution is incubated in a thermostatic condition. And then a signal reagent is added to the reaction container when the B/F process is finished. Optical detection of the reaction container is executed after the signal reagent is added to the reaction container for incubation for a certain time. The optical detection may be performed immediately after adding the signal reagent without incubation. For example, a chemiluminescence test based on electrochemical luminescence or flash system is shown in FIG. 3.

(2) Two-Step, One-B/F Protocol

Referring to FIG. 4, a first reagent (which may include several kinds of compositions) and a sample are added to a reaction container and mixed to form a reaction solution, and the reaction container is incubated in a thermostatic condition for a certain time. Thereafter, a second reagent (which may include several kinds of compositions) is added to the reaction container and mixed with the first reagent and the sample in the reaction solution. The mixed reaction container is incubated in a thermostatic condition for a certain time, and B/F is executed after incubation. A signal reagent is added to the reaction container when B/F is finished. After the reaction container with the signal reagent is incubated in a thermostatic condition for a certain time, optical detection is executed. As mentioned before, some kinds of tests may execute an optical detection directly after adding the signal reagent without any incubation.

(3) Two-Step, Two-B/F Protocol

Referring to FIG. 5, a first reagent (which may include several kinds of compositions) and a sample are added to a reaction container and mixed to form a reaction solution. The B/F process is executed after the reaction solution is incubated in a thermostatic condition for a certain time. Thereafter, a second reagent (which may include several kinds of compositions) is added to the reaction container and mixed with the reaction solution containing the first reagent and the sample. After the reaction container is incubated in a thermostatic condition for a certain time, the B/F process is executed. A signal reagent is then added to the reaction container. After the reaction container with the signal reagent is incubated in a thermostatic condition for a certain time, an optical detection is executed. As mentioned before, some kinds of tests may execute an optical detection directly after adding the signal reagent without any incubation.

During an immunoassay analysis, it is important for an automatic immunoassay analyzer to dispense samples and reagents automatically and precisely. With an increase of test samples, the samples or/and the reagents often need to be dispensed quickly. Because the immunoassay analyzer has a limited space, different units of the immunoassay analyzer need to cooperate closely so as to achieve the highest test throughput. The test throughput is defined as a quantity of test results reported using the immunoassay analyzer in a unit time.

For automated test technologies, a blood analyzer usually uses a puncture sample needle to dispense a sample, and then the sample flows to a reagent dispensing station by a fluid system, so as to realize a reagent dispensing. This analyzer is limited to analyzing a few kinds of reagents, and the fluid system of the analyzer is complex in structure, high in cost and low in reliability.

A biochemical analyzer usually includes a reaction wheel, and the reaction wheel is arranged with a sample dispensing station, a reagent dispensing station, an agitating station, and a washing station. A sample probe is arranged near the reaction wheel for dispensing samples to a reaction container in the sample dispensing station, and a reagent probe is arranged near the reaction wheel for dispensing reagents to a reaction container in the reagent dispensing station. However, these dispensing mechanisms of the sample probe and the reagent probe require the reaction wheel to move regularly according to a specific period, and the reaction wheel must be stationary when the samples or reagents are dispensed to the reaction container. Therefore, a movement of the reaction wheel is limited, and a flexible test process is limited also. In addition, the sample probe and/or the reagent probe must move to pass the reaction wheel, which limits an arrangement of the sample probe and/or the reagent probe, and increases difficulties to manufacture a biochemical analyzer.

In addition to using a dispensing device and method similar to those of the biochemical analyzer, the immunoassay analyzer arranges a dispensing station on a periphery of an incubation unit, so as to realize a sample dispensing and a reagent dispensing at the dispensing station. Then a reaction container dispensed with the sample and the reagent is transferred to the incubation unit. Therefore, a sample probe and a reagent probe must be arranged around the dispensing station, and the sample and the reagent are dispensed at different times. As such, this test process results in low test throughput. Furthermore, after the reaction container dispensed with the sample and a first reagent is transferred to the incubation reaction unit for incubation for a while, the reaction container must be transferred to a dispensing station for dispensing a second reagent. After that, the reaction container is again transferred to the incubation reaction unit for incubation. Therefore, the test process is complex and transfer actions are added.

In one embodiment, an immunoassay analyzer includes a first dispensing station and a second dispensing station. The first dispensing station is arranged on a periphery of a holding unit, and the second dispensing station is arranged on the holding unit. The immunoassay analyzer also includes a dispensing unit that dispenses a first reaction component at the first dispensing station and dispenses a second reaction component at the second dispensing station. The holding unit may be a reaction wheel, which provides a reaction place for reaction components. The first reaction component and the second reaction component may include the following four combinations:

(1) the first reaction component is a sample, and the second reaction component is a reagent;

(2) the first reaction component is a sample and a first reagent, and the second reaction component is a second reagent;

(3) the first reaction component is a reagent, and the second reaction component is a sample; or

(4) the first reaction component is a first reagent, and the second reaction component is a sample and a second reagent.

The first reaction component and the second reaction component can be determined according to a specific process of immunoassay analysis, where the terms “first” and “second” are used for differentiation and do not have any limitation; therefore, the first reaction component and the second reaction component may be jointly named as reaction components. In the present disclosure, the first dispensing station and the second dispensing station are arranged separately, so the first reaction component can be dispensed while other operations are performed in parallel by the holding unit. At the same time, the dispensing unit is not restricted to being arranged around the holding unit, so restrictions and interference of space can be avoided.

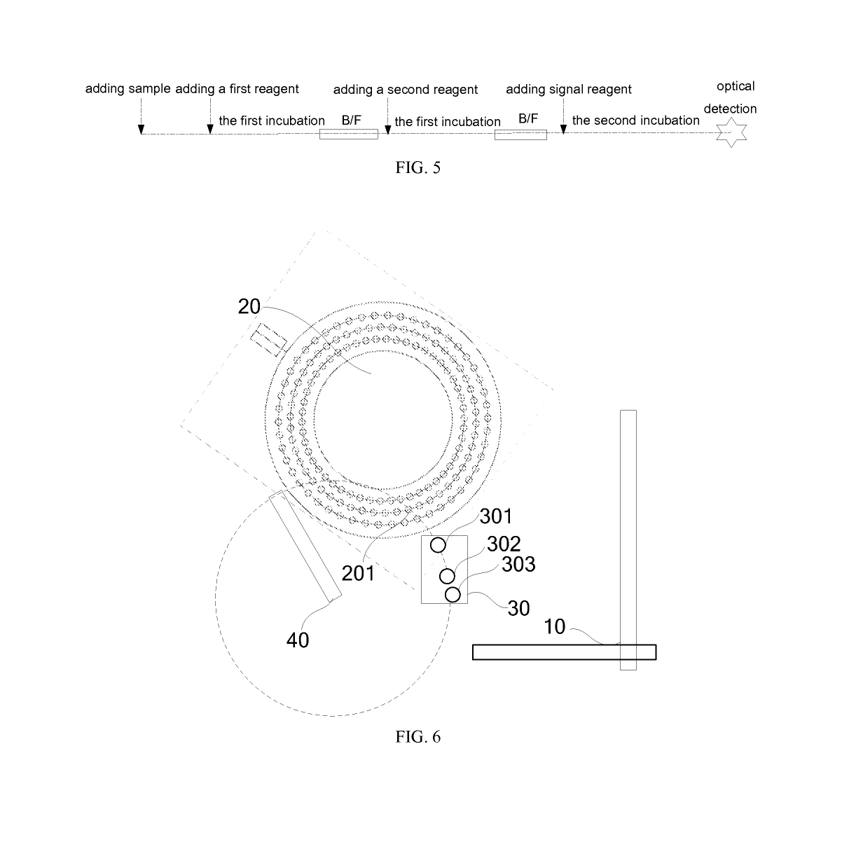

Referring to FIG. 6, the present embodiment provides a dispensing device, including a transfer unit 10, a holding unit 20, a first dispensing station 30, a second dispensing station 201, a dispensing unit 40 and a control unit (not shown).

The transfer unit 10 can be used to transfer a reaction container, and a transfer path of the transfer unit 10 can pass the holding unit 20, which can hold the reaction container. The first dispensing station 30 is arranged on a periphery of the holding unit 20, and the transfer path of the transfer unit 10 can also pass the first dispensing station 30. Therefore, the transfer unit 10 can transfer the reaction container between the holding unit 20 and the first dispensing station 30. The second dispensing station 201 is arranged on the holding unit 20, and the transfer unit 10 may transfer the reaction container between the first dispensing station 30 and the holding unit 20. In an embodiment, the second dispensing station 201 is arranged on a transfer path of the holding unit 20. In this case, when the transfer unit 10 transfers the reaction container between the first dispensing station 30 and the second dispensing station 201, the second dispensing station 201 can be a transfer station of the holding unit 20, where the transfer station is a position of the holding unit 20 at which the transfer unit 10 can transfer the reaction container into or out of the holding unit 20.

In another case, the transfer station of the holding unit 20 is arranged in a different location from the second dispensing station 201. That is, the transfer unit 10 transfers the reaction container from the first dispensing station 30 to the transfer station, and then the holding unit 20 brings the reaction container from the transfer station to the second dispensing station 201. An operation path of the dispensing unit 40 passes the first dispensing station 30 and the second dispensing station 201, and the dispensing unit 40 dispenses a first reaction component to the reaction container located at the first dispensing station 30, and dispenses a second component to the reaction container located at the second dispensing station 201.

The holding unit 20 is a ring structure. The holding unit 20 can be a disc transfer mechanism or an orbital transfer mechanism, and the holding unit 20 includes at least one ring. When the holding unit 20 includes more than one ring, all the rings may be driven together to reduce drive mechanisms so as to reduce cost, or each ring may be driven separately. When the holding unit 20 includes three rings, then an outer ring may be driven separately, while a middle ring and an inner ring are driven together, or the outer ring, middle ring and inner ring may be driven separately so as to realize flexible control. One or more positions, such as holes or slots, can be arranged on each ring of the holding unit 20, where those positions can be used for holding the reaction container and transferring the reaction container to a predetermined transfer station, such that the reaction container can be operated by other units of the device.

The first dispensing station 30 can include a dispensing position 301 and a dilution position 302 (303). The first reaction component can be dispensed to the reaction container held at the dispensing position 301 of the first dispensing station 30, and a diluted sample can be drawn by the first dispensing station 30 from the reaction container held at the dilution position 302 (303). The dispensing position 301 and the dilution position 302 (303) are both located on the operation path of the same dispensing unit 40 and the transfer path of the transfer unit 10.

The first dispensing station 30 is arranged on the periphery of the holding unit 20, so a movement of the holding unit 20 is not restricted by the first dispensing station 30. When the dispensing unit 40 dispenses the first reaction component to the reaction container located at the first dispensing station 30, the holding unit 20 can transfer the reaction container or perform other operations. As such, the holding unit 20 can transfer the reaction container more flexibly, so as to improve test efficiency and test throughput of the whole instrument.

The second dispensing station 201 is also arranged on the operation path of the dispensing unit 40, and the dispensing unit 40 can dispense the second reaction component to the reaction container located at the second dispensing station 201. In the present embodiment, the second dispensing station 201 is arranged along the transfer path of the holding unit 20, and may be regarded as the transfer station of the holding unit 20. When the reaction container of the holding unit 20 needs to receive dispensed reagents many times, the holding unit 20 may transfer the reaction container to the second dispensing station 201, to achieve the dispensing of reagents without transferring the reaction container out of the holding unit 20. As such, additional actions of the transfer unit 10 are reduced, testing procedures and processes are simplified, the reliability of the instrument is improved, and the service life of the instrument is prolonged.

The control unit may be respectively coupled to the transfer unit 10, the holding unit 20 and the dispensing unit 40. The control unit may control the transfer unit 10 to transfer the reaction container at least between the first dispensing station 30 and the holding unit 20, may control the holding unit 20 to transfer the reaction container, and may control the dispensing unit 40 to dispense the first reaction component to the reaction container located at the first dispensing station 30 and to dispense the second reaction component to the reaction container located at the second dispensing station 201. The control unit can also be used to control a sample analysis of the dispensing device. The control unit may control analysis steps of the sample automatically, and the control unit may also perform the analysis steps after receiving control orders input by an operator.

In an example, the first dispensing station 30 may be used to dispense a sample, and the second dispensing station 201 may be used to dispense a reagent. Alternatively, the first dispensing station 30 may dispense the reagent, and the second dispensing station 201 may dispense the sample.

Furthermore, for the present dispensing device, the first dispensing station 30 is arranged on the periphery of the holding unit 20, and the second dispensing station 201 is arranged on the holding unit 20; therefore, the dispensing unit 40 is not restricted to being arranged around the holding unit 20. Because the first dispensing station 30 and the second dispensing station 201 are arranged separately, space restrictions and interference with the dispensing unit 40 are avoided. In addition, dispensing of both the first reaction component and the second reaction component is performed by the dispensing unit 40, thereby reducing a production cost of the immunoassay analyzer. The present dispensing device can reduce the product cost and increase the test throughput.

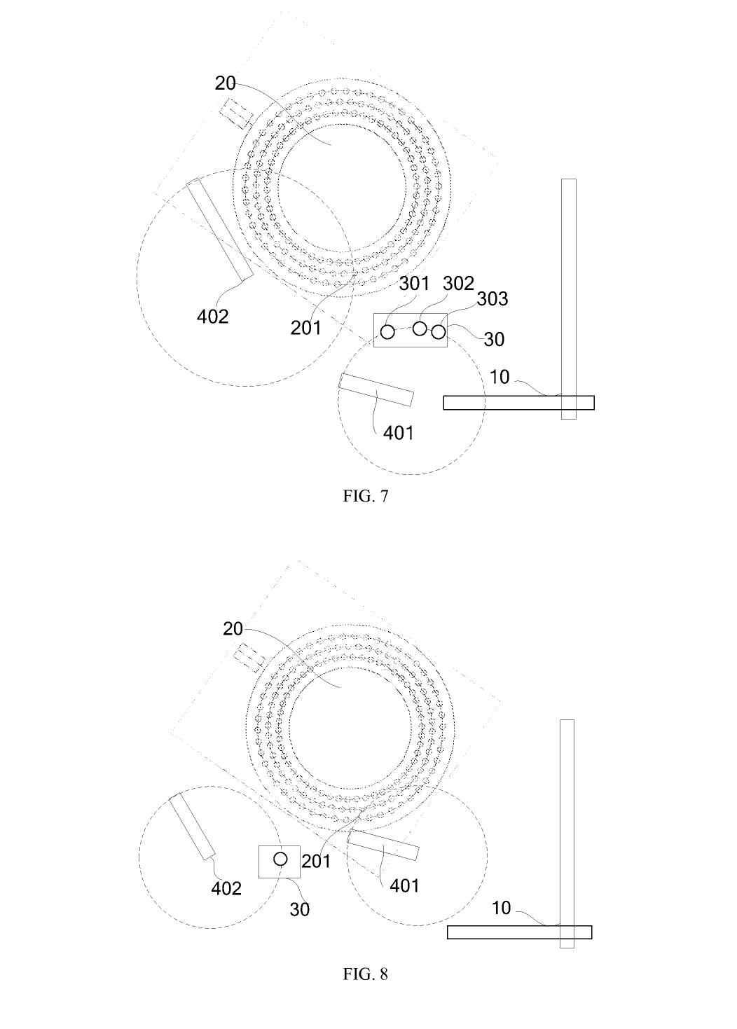

Referring to FIG. 7, the present embodiment also provides another dispensing device including a first dispensing unit 401 and a second dispensing unit 402. An operation path of the first dispensing unit 401 passes the first dispensing station 30 for dispensing the first reaction component to the reaction container. An operation path of the second dispensing unit 402 passes the second dispensing station 201 for dispensing the second reaction component to the reaction container. FIG. 8 shows a structure of this dispensing device.

In an example, the second dispensing unit 402 dispenses the reagent to the reaction container located at the first dispensing station 30, then the transfer unit 10 transfers the reaction container to the second dispensing station 201, and the first dispensing unit 401 dispenses the sample to the reaction container located at the second dispensing station 201.

In some other examples, the second dispensing unit 402 may dispense the reagent to the reaction container located at the second dispensing station 201, then the transfer unit 10 may transfer the reaction container to the first dispensing station 30, and the first dispensing unit 401 may dispense the sample to the reaction container located at the first dispensing station 30.

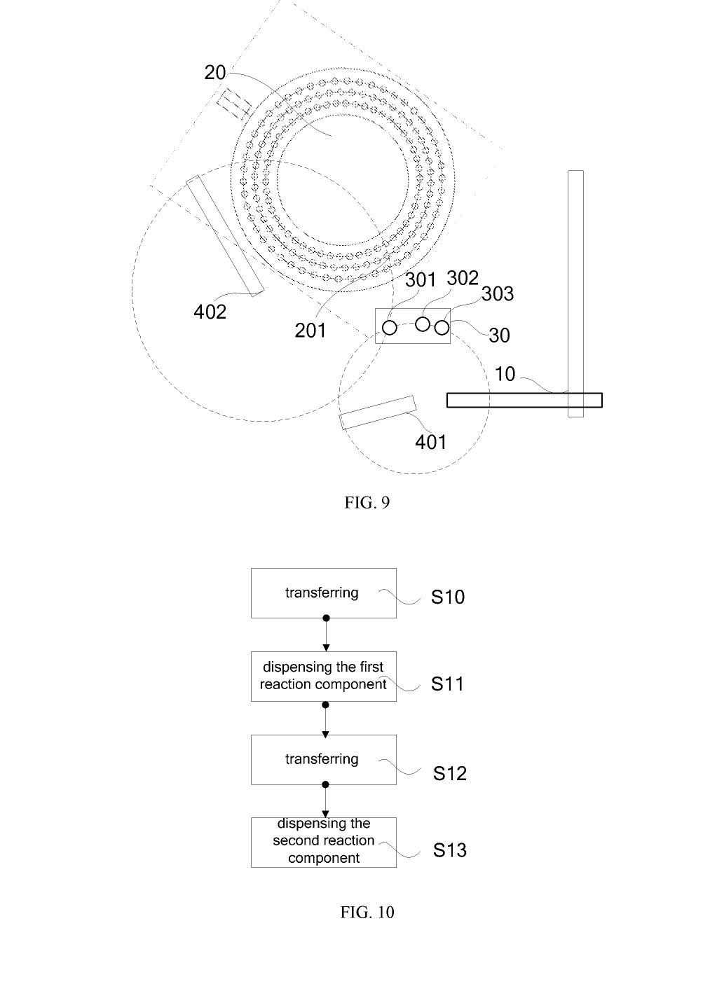

Specifically, the dispensing unit and the dispensing station need not have a one-to-one relationship. The relationship can be set according to specific need. Referring to FIG. 9, the operation path of the second dispensing unit 402 can also pass the first dispensing station 30, that is, the operation path of the second dispensing unit passes the first dispensing station 30 and the second dispensing station 201. So the second dispensing unit 402 may not only dispense the first reaction component at the first dispensing station 30, but may also dispense the second reaction component at the second dispensing station 201. In a specific example, the first dispensing unit 401 may first dispense the sample to the reaction container located at the first dispensing station 30; the second dispensing unit 402 may then dispense the reagent to the reaction container located at the first dispensing station 30; the transfer unit 10 may subsequently transfer the reaction container to the second dispensing station 201. If any reagent needs to be dispensed to the reaction container in a subsequent test process, the holding unit 20 may transfer the reaction container to the second dispensing station 201, and the second dispensing unit 402 can dispense the reagent to the reaction container located at the second dispensing station 201.

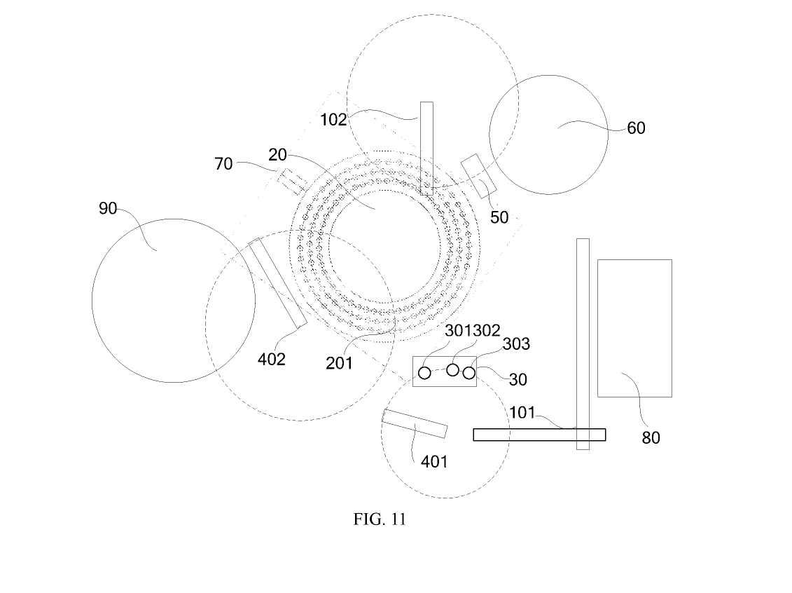

Referring to FIG. 10, the present embodiment also provides a dispensing method including the following steps S10-S13.

In step S10, a control unit may control a reaction container to be transferred by a transfer unit to a first dispensing station which is located on a periphery of a holding unit. In a specific operation process, the reaction container may also be transferred to the first dispensing station by a dedicated transfer mechanism, such as a transfer rack or a transfer orbit.

In step S11, the control unit may control a dispensing unit to dispense a first reaction component to the reaction container which is located at the first dispensing station.

In step S12, the control unit may control the transfer unit to transfer the reaction container held at the first dispensing station to a second dispensing station arranged on the holding unit. That is, the second dispensing station can be arranged on a transfer path of the holding unit, and thus the transfer unit may transfer the reaction container held at the first dispensing station to the second dispensing station on the holding unit. At this point, the second dispensing station can be a transfer station of the holding unit, where the transfer station is a position of the holding unit at which the transfer unit transfers the reaction container into the holding unit or out of the holding unit. In another case, the transfer station of the holding unit and the second dispensing station may be arranged at two different locations; in this case, the transfer unit may transfer the reaction container to the transfer station, and then the holding unit may transfer the reaction container to the second dispensing station.

In step S13, the control unit can control the dispensing unit to dispense a second reaction component to the reaction container which is located at the second dispensing station.

In another embodiment, the dispensing unit involved in the steps S11 and S13 can include a first dispensing unit and a second dispensing unit. In step S11, the first dispensing unit may dispense the first reaction component to the reaction container which is located at the first dispensing station, and in step S13, the second dispensing unit may dispense the second reaction component to the reaction container which is located at the second dispensing station.

In a specific embodiment, the first reaction component of step S11 can be a sample, and the dispensing method can further include a dilution step when there is a need to dilute a sample having a high concentration. Specifically, the transfer unit may transfer the reaction container with the sample from the first dispensing station to the second dispensing station on the holding unit; the second dispensing unit may add a diluent to the reaction container located at the second dispensing station; and then the transfer unit may transfer the reaction container with a diluted sample to a dilution position of the first dispensing station, such that the first dispensing unit may draw the diluted sample for analysis. In this case, the diluted sample is regarded as a test sample, and the first dispensing unit draws the diluted sample at the dilution position for analysis.

The present embodiment provides an immunoassay analyzer which may include the above-described dispensing device.

Referring to FIG. 11, the immunoassay analyzer can include a first transfer unit 101, a second transfer unit 102, a holding unit 20, a first dispensing station 30, a second dispensing station 201, a first dispensing unit 401, a second dispensing unit 402, an agitating unit 50, a wash unit 60, an optical detection unit 70, a reaction container supply unit 80, a reagent container 90 and a control unit (not shown).

The reaction container supply unit 80, the first dispensing station 30 and the holding unit 20 can be arranged along a transfer path of the first transfer unit 101, and the first transfer unit 101 can transfer a reaction container between the reaction container supply unit 80, the first dispensing station 30 and the holding unit 20.

The holding unit 20 is used for holding the reaction containers.

The first dispensing station 30 may be arranged on a periphery of the holding unit 20, and the second dispensing station 201 may be arranged on the holding unit 20. An operation path of the first dispensing unit 401 can pass the first dispensing station 30 for dispensing a first reaction component to the reaction container located at the first dispensing station 30. An operation path of the second dispensing unit 402 can pass the second dispensing station 201 for dispensing a second reaction component to the reaction container located at the second dispensing station 201. In a specific embodiment, the first reaction component is a sample, and the second reaction component is a reagent.

The agitating unit 50 may be arranged on the periphery of the holding unit 20, and may be used to agitate a reaction solution in the reaction container. The agitating unit 50 is an optional structure for the immunoassay analyzer, and the sample and the reagent do not need to be agitated in some embodiments.

The wash unit 60 may be arranged on the periphery of the holding unit 20, and may be used for removing unbound components in the reaction container. A signal reagent can then be added to the reaction container to react with reaction complexes formed by the first reaction component and the second reaction component within the reaction container to generate an optical signal.

The holding unit 20, the agitating unit 50 and the wash unit 60 can be arranged on a transfer path of the second transfer unit 102, and the second transfer unit 102 can transfer the reaction container between the holding unit 20, the agitating unit 50 and the wash unit 60.

The optical detection unit 70 may be arranged on the periphery of the holding unit 20, and can be used for detecting an analyte within the reaction container located at the holding unit 20. The optical detection unit 70 may be a photometer, which detects a concentration of the analyte by detecting an intensity of the optical signal.

The reaction container supply unit 80 can be used for supplying the reaction containers for the immunoassay analyzer. The first transfer unit 101 can transfer a new reaction container from the reaction container supply unit 80 to the first dispensing station 30.

The reagent container 90 is used for holding reagent, for example, reagent bottles. The second dispensing unit 402 may dispense the reagent from the reagent container 90 to the reaction container located at the second dispensing station 201.

The control unit may be respectively coupled to the first transfer unit 101, the second transfer unit 102, the holding unit 20, the first dispensing unit 401 and the second dispensing unit 402. The control unit can control the first transfer unit 101 to transfer the reaction container between the reaction container supply unit 80, the first dispensing station 30 and the holding unit 20; can control the second transfer unit 102 to transfer the reaction container between the holding unit 20, the agitating unit 50 and the wash unit 60; can control the holding unit 20 to transfer the reaction container; and can control the first dispensing unit 401 and the second dispensing unit 402 to respectively dispense the first reaction component and the second reaction component to the reaction container located at the first dispensing station 30 and the second dispensing station 201. The control unit can be used to control the immunoassay analyzer to perform a sample analysis. The control unit may control analysis steps of the sample automatically, and the control unit may also perform the analysis steps after receiving control orders input by an operator.

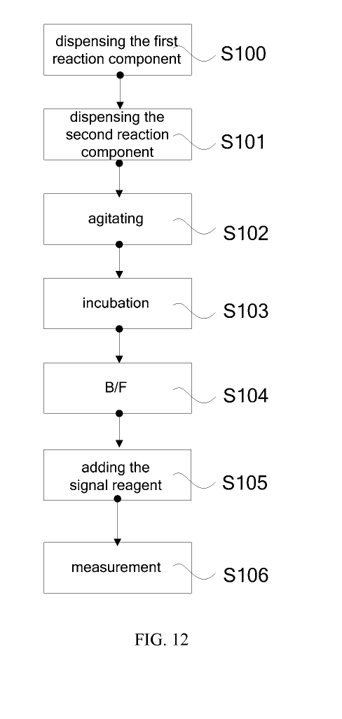

Referring to FIG. 12, the present embodiment also provides an immunoassay analysis method including the following steps S100-S106.

In step S100, a control unit may control a transfer unit to transfer a new reaction container from a reaction container supply unit to a dispensing position of a first dispensing station. The first dispensing station can be located on a periphery of a holding unit. The control unit may then control a dispensing unit to dispense a first reaction component to the reaction container which is located at the dispensing position of the first dispensing station. In the present embodiment, the first reaction component is a sample, and in another embodiment, the first reaction component may be a reagent.

In step S101, the holding unit transfers a position without a reaction container to a second dispensing station; after the first reaction component is added into the reaction container, the control unit may control the transfer unit to transfer the reaction container with the first reaction component to the second dispensing station, which is located at the holding unit. The control unit may control the dispensing unit to dispense a second reaction component to the reaction container which is located at the second dispensing station.

In the present embodiment, the second reaction component is the reagent. Furthermore, the dispensing unit can include a first dispensing unit and a second dispensing unit. The first dispensing unit can dispense the sample to the reaction container which is located at the first dispensing station, and the second dispensing unit can dispense the reagent from the reagent container to the reaction container located at the second dispensing station.

In step S102, the control unit may control the holding unit to transfer the reaction container with the reagent to a transfer station. The transfer unit can transfer the reaction container to an agitating unit for agitating, and after the agitating can transfer the reaction container back to the holding unit.

The transfer station is a position of the holding unit at which the transfer unit transfers the reaction container into the holding unit or out of the holding unit. In the present embodiment, the transfer unit may include a first transfer unit and a second transfer unit. The first transfer unit can transfer the reaction container between the reaction container supply unit, the first dispensing station and the transfer station of the holding unit. The second transfer unit can transfer the reaction container between the transfer station of the holding unit and the agitating unit. In some embodiments, the sample and the reagent do not need to be agitated, so step S102 may be canceled.

In step S103, the control unit can control the holding unit to transfer the reaction container after agitating to an incubation position for incubation. In the present embodiment, the holding unit may have three rings, i.e., an outer ring, a middle ring and an inner ring. After agitating is finished, the second transfer unit may transfer the reaction container back to any ring of the three rings for incubation according to practical requirements.

In step S104, after the first reaction component and the second reaction component are incubated within the reaction container for an incubation time, the control unit may control the holding unit to transfer the reaction container to the transfer station, and then control the second transfer unit to transfer the reaction container to the wash unit for removing unbound components within the reaction container.

In step S105, a signal reagent can be added into the reaction container after the reaction container is washed in the wash unit, and then the reaction container containing the signal reagent may be transferred back to the holding unit. In the present embodiment, the second transfer unit can also transfer the reaction container between the holding unit and the wash unit.

In step S106, the control unit may control the holding unit to transfer the reaction container with the signal reagent to a detection station of the holding unit. An optical detection unit is arranged on the periphery of the holding unit for detecting an analyte within the reaction container.

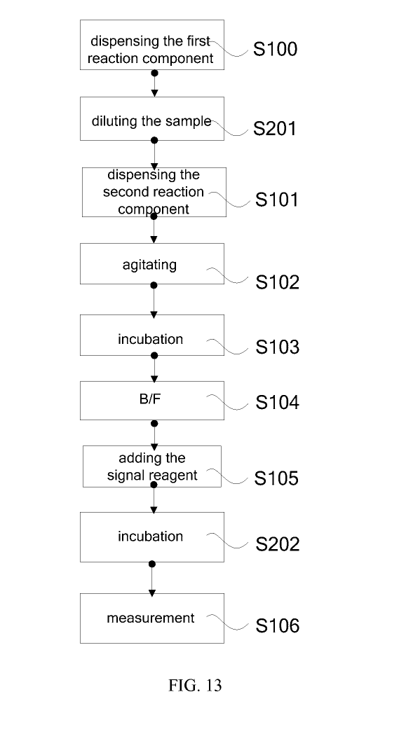

Referring to FIG. 13, when the sample must be diluted due to a high concentration of the analyte in the sample, an immunoassay method provided in another embodiment can further include the following step S201 between the steps S100 and S101.

In step S201, the first transfer unit may transfer the reaction container with the sample located at the first dispensing station to the second dispensing station on the holding unit, and then the second dispensing unit may dispense a diluent to the reaction container located at the second dispensing station. The holding unit can transfer the reaction container to the transfer station of the holding unit, and then the second transfer unit can transfer the reaction container from the transfer station to the agitating unit for agitating. After the reaction container is agitated, the second transfer unit may transfer the reaction container back to the transfer station, where the holding unit may transfer the reaction container to the second dispensing station. The first transfer unit can transfer the reaction container with a diluted sample to a dilution position of the first dispensing station, such that the first dispensing unit can draw a diluted sample at the dilution position for analysis. Therefore, the diluted sample is regarded as a test sample. The first dispensing unit may draw the diluted sample at the dilution position, and then dispense the diluted sample to the new reaction container which is located at the dispensing position of the first dispensing station. The first transfer unit may transfer the new reaction container to the transfer station, and then the holding unit may transfer the reaction container to the second dispensing station, such that the second dispensing unit can add the reagent for future test.

In some embodiments, after the second dispensing unit dispenses the diluents to the reaction container which is located at the second dispensing station in step S201, there is no need to agitate the reaction container, and the first transfer unit can transfer the reaction container to the dilution position of the first dispensing station directly.

In another embodiment, the method may also include the following step S202 between the steps S105 and S106.

In step S202, the holding unit may transfer the reaction container with the signal reagent to the incubation position for incubation. Correspondingly, the holding unit may transfer the reaction container after the incubation to the detection station of the holding unit in step S106, such that the optical detection unit located on the periphery of the holding unit can detect the analyte of the reaction container.

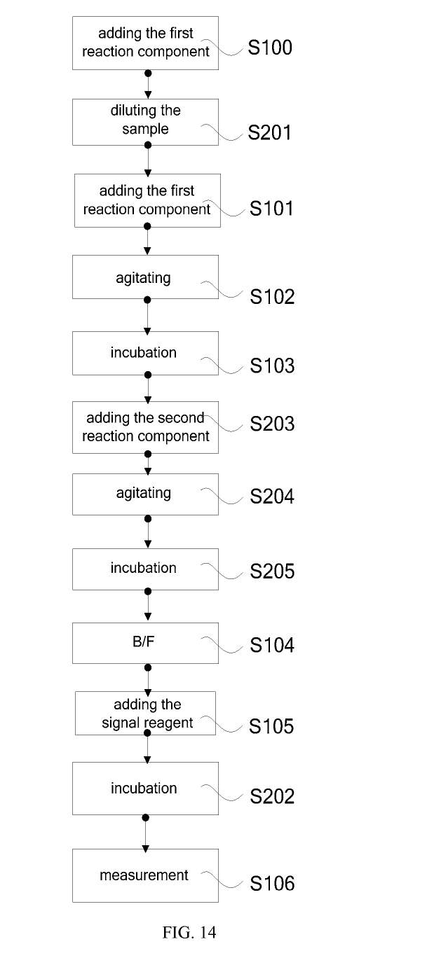

The immunoassay methods described above are a one-step protocol. Referring to FIG. 14, the embodiment also provides another immunoassay method based on a two-step, one-B/F protocol, where the method may further include extra steps S203-S205 between steps S103 and S104 of the one-step protocol.

In step S203, the holding unit transfers the reaction container that holds the first reaction component and the second reaction component for the incubation time in step S103 to the second dispensing station, where the dispensing unit dispenses a third reaction component to the reaction container. The third reaction component is another reagent, which was not dispensed when dispensing the second reaction component at step S101.

In step S204, the holding unit may transfer the reaction container with the third reaction component to the transfer station of the holding unit, the transfer unit may transfer the reaction container located at the transfer station to the agitating unit for agitating, and then the transfer unit may transfer the reaction container after agitating back to the holding unit.

In step S205, the holding unit may transfer the reaction container after agitating to the incubation position for incubation.

In another immunoassay method, a wash step can be performed between steps S103 and S203 based on the two-step, one-B/F protocol, so as to form a two-step, two-B/F protocol.

The transfer unit, the holding unit and the dispensing unit can be controlled by the control unit in the immunoassay methods provided in this disclosure.

For the dispensing devices, immunoassay analyzers and methods thereof in various embodiments of this disclosure, the sample and the reagent are dispensed in the first dispensing station and the second dispensing station. When the first reaction component is dispensed in the first dispensing station, the holding unit is not affected by dispensing the first reaction component, and the reaction container can be transferred to perform other operations. Therefore, the dispensing of the first reaction component and other operations of the holding unit can be performed at the same time, thereby improving the test efficiency. When the reagent needs to be dispensed into the reaction container located at the holding unit, to simplify the test process, the holding unit may directly transfer the reaction container to the second dispensing station for dispensing the reagent, and there is no need to transfer the reaction container out of the holding unit. In addition, the dispensing unit is not restricted to being arranged around the holding unit, while the first dispensing station and the second dispensing station are arranged separately, so that space restrictions and interference with the dispensing units are avoided. When using the dispensing devices and methods of this disclosure, the test throughput can be improved while cost can be reduced. The immunoassay analyzers described here are suitable for various test protocols including the one-step protocol; the two-step, one-B/F protocol; and the two-step, two-B/F protocol.

The sample and reagent dispensing devices and methods in various embodiments of this disclosure can be applied not only to the immunoassay analyzer, but also to other apparatus which need to dispense the sample and reagent. For example, the sample and reagent dispensing devices and methods may be applied to a biochemical analyzer through some modifications.

The above-described contents are detailed with specific and preferred embodiments for the present invention. The implementation of the present invention is not to be limited to these illustrations. For one of ordinary skill in the art, variations and equivalents having the same effects and applications can be made without departing from the spirit of the present invention and are to be considered as belonging to the scope of the present invention.

What is claimed is:

1. A dispensing device, comprising:
a holding unit that holds and transfers one or more reaction containers along a transfer path of the holding unit;
a first dispensing station that is arranged on a periphery of the holding unit;
a second dispensing station that is arranged on the holding unit;
a transfer unit that transfers the one or more reaction containers between the first dispensing station and the holding unit on which the second dispensing station is arranged;
a dispensing unit that dispenses a first reaction component to the reaction containers located at the first dispensing station at a dispensing position of the first dispensing station and dispenses a second reaction component to the reaction containers located at the second dispensing station; and
a control unit coupled to the transfer unit, the holding unit and the dispensing unit, wherein:
the control unit controls the transfer unit to transfer the one or more reaction containers between the first dispensing station and the holding unit on which the second dispensing station is arranged, and controls the dispensing unit to dispense the first reaction component to the reaction containers located at the first dispensing station and dispense the second reaction component to the reaction containers located at the second dispensing station.

2. The dispensing device of claim 1, wherein the first dispensing station further comprises a dilution position where a diluted sample is drawn.

3. The dispensing device of claim 1, wherein the dispensing unit comprises a first dispensing unit and a second dispensing unit, the first dispensing unit dispenses the first reaction component to the reaction containers located at the first dispensing station, and the second dispensing unit dispenses the second reaction component to the reaction containers located at the second dispensing station.

4. The dispensing device of claim 1, wherein the holding unit is a disc transfer mechanism or an orbital transfer mechanism that is a ring structure comprising one or more rings.

5. A dispensing method, comprising:
transferring by a transfer unit one or more reaction containers to a dispensing position of a first dispensing station which is arranged on a periphery of a holding unit;
dispensing by a dispensing unit a first reaction component to the one or more reaction containers located at the first dispensing station;
transferring by the transfer unit the one or more reaction containers from the first dispensing station to a second dispensing station which is arranged on the holding unit; and
dispensing by the dispensing unit a second reaction component to the reaction containers located at the second dispensing station.

6. The dispensing method of claim 5, wherein the first reaction component is a sample and the second reaction component is a reagent, and wherein the first dispensing station further comprises a dilution position;
after the transfer unit transfers the one or more reaction containers to the second dispensing station on the holding unit, and before dispensing the second reaction component to the reaction containers located at the second dispensing station, the method further comprises diluting the sample, wherein diluting the sample comprises:
dispensing by the dispensing unit a diluent to the reaction containers located at the second dispensing station to obtain a diluted sample, and
transferring by the transfer unit the reaction containers containing the diluted sample to the dilution position of the first dispensing station, such that the diluted sample is drawn for analysis.

7. An immunoassay analyzer, comprising,
a dispensing device, comprising:
a holding unit that holds and transfers one or more reaction containers along a transfer path of the holding unit;
a first dispensing station that is arranged on a periphery of the holding unit;
a second dispensing station that is arranged on the holding unit;
a transfer unit that transfers the one or more reaction containers between the first dispensing station and the holding unit on which the second dispensing station is arranged;
a dispensing unit that dispenses a first reaction component to the reaction containers located at the first dispensing station at a dispensing position of the first dispensing station and dispenses a second reaction component to the reaction containers located at the second dispensing station; and
a control unit coupled to the transfer unit, the holding unit and the dispensing unit, wherein:
the control unit controls the transfer unit to transfer the one or more reaction containers between the first dispensing station and the holding unit on which the second dispensing station is arranged, and controls the dispensing unit to dispense the first reaction component to the reaction containers located at the first dispensing station and dispense the second reaction component to the reaction containers located at the second dispensing station;
a wash unit arranged on the periphery of the holding unit, the wash unit removes an unbound component from the one or more reaction containers and adds a signal reagent which can react with reaction complexes formed by the first reaction component and the second reaction component in the reaction containers for generating an optical signal; and
an optical detection unit, which is arranged on the periphery of the holding unit and detects the optical signal from the reaction containers located at the holding unit.

8. The analyzer of claim 7, wherein the transfer unit comprises a first transfer unit and a second transfer unit, and the control unit controls the first transfer unit to transfer the one or more reaction containers between the first dispensing station and the holding unit, and the control unit controls the second transfer unit to transfer the one or more reaction containers between the holding unit and the wash unit.

9. The analyzer of claim 8, further comprising,
a reaction container supply unit, which supplies the reaction containers; and
a reagent container, which holds reagents;
wherein the control unit further controls the first transfer unit to transfer the one or more reaction containers between the reaction container supply unit and the first dispensing station.

10. An immunoassay method, comprising:
transferring by a transfer unit a reaction container to a first dispensing station, which is arranged on a periphery of a holding unit;
dispensing by a dispensing unit a first reaction component to the reaction container located at the first dispensing station;
transferring by the transfer unit the reaction container from the first dispensing station to a second dispensing station arranged on the holding unit;
dispensing by the dispensing unit a second reaction component to the reaction container located at the second dispensing station;
transferring by the holding unit the reaction container that holds the first reaction component and the second reaction component to a transfer station of the holding unit for an incubation time;
transferring by the transfer unit the reaction container from the transfer station to a wash unit for removing an unbound component from the reaction container;
adding a signal reagent that reacts with reaction complexes formed by the first reaction component and the second reaction component within the reaction container to generate an optical signal; and
transferring by the holding unit the reaction container with the signal reagent to a detection station of the holding unit;
wherein the optical signal from the reaction container is detected by an optical detection unit arranged on the periphery of the holding unit.
